(19) United States
(12) Patent Application Publication
(10) Pub. No.: US 2011/0066029 A1
(43) Pub. Date: Mar. 17, 2011

(54) Electromagnetic Medical Device

(75) Inventors: SuPing Lyu, Maple Grove, MN (US); Micheal R. Neidert, Salthill (IE); Christipher M. Hobot, Tonka Bay, MN (US); James Louis Schley, Coon Rapids, MN (US); Kenneth C. Gardeski, Plymouth, MN (US)

(73) Assignee: Medtronic, Inc., Minneapolis, MN (US)

(21) Appl. No.: 12/557,653

(22) Filed: Sep. 11, 2009

(51) Int. Cl. A61B 5/05

(52) U.S. Cl. 600/424

(57) ABSTRACT

An insertable or implantable medical device includes an elongated member having a proximal end, a distal end, at least one conductive coil near the distal end, and electrical conductors which carry current from the coil towards the proximal end. The coil surrounds or is surrounded by a flexible magnetic polymeric composite.

### FIELD

This invention relates to catheters and other insertable or implantable medical devices.

### BACKGROUND

Various specialized insertable or implantable medical devices, including catheters (e.g., ablation catheters, electrophysiological diagnostic catheters, pressure monitoring catheters and delivery catheters), leads (e.g., cardiac and neurological leads) and other elongated medical devices, are sometimes equipped with location sensors for determining the location of the device within a patient. Multiple location sensors may be arrayed along a distal segment of an elongated medical device to provide a more intuitive indication of the device location than would be provided by a single location sensor.

One type of location sensor employs an electromagnetic coil in which current is induced by an externally applied electromagnetic field. The location of the coil relative to the field may be determined by measuring the induced current and performing appropriate calculations. Some elongated insertable or implantable medical devices include an extruded polymeric covering, lumen or tube, and in such devices an electromagnetic location sensor may be formed by wrapping wire (e.g., copper wire) in a helical coil around the covering, lumen or tube. Other devices may include an electromagnetic location sensor formed by wrapping wire in a helical coil around a core made from solid or powdered magnetically permeable material.

### SUMMARY

Electromagnetic location sensors formed by wrapping wire around a polymeric covering, lumen or tube do not receive the amplification benefit of being wrapped around a high permeability core. This can limit the induced current signal and impair sensitivity, signal to noise ratio or accuracy. Electromagnetic location sensors formed by wrapping wire around a solid or powdered magnetically permeable core may have greater magnetic permeability than sensors formed around a polymeric covering, lumen or tube, but also have high stiffness. This can make it difficult to insert or implant a medical device equipped with such sensors, especially if the medical device also includes other inflexible or not very flexible elements such as electrical conductors, guide wires or steering wires. Sensors formed using such cores may provide improved results if the core is lengthened appreciably (viz., in the axial direction) so that it extends beyond the wire coil length, but this may further limit flexibility compared to a sensor made on a shorter core.

The present invention provides, in one aspect, an insertable or implantable medical device comprising an elongated member having a proximal end, a distal end, at least one conductive coil near the distal end, and electrical conductors which carry current from the coil towards the proximal end, wherein the coil surrounds or is surrounded by a flexible magnetic polymeric composite.

The invention provides, in another aspect, a location sensor bobbin comprising a conductive coil surrounding or surrounded by a flexible magnetic polymeric composite, the bobbin being hollow and being sized and shaped to fit on or into an elongated insertable or implantable medical device.

The invention provides, in another aspect, a method for making an insertable or implantable medical device, which method comprises forming an elongated member having a proximal end and a distal end, forming at least one conductive coil surrounding or surrounded by a flexible magnetic polymeric composite near the distal end, and connecting electrical conductors to the coil to carry current from the coil towards the proximal end

The invention provides, in another aspect, a method for locating an elongated insertable or implantable medical device in a patient, which method comprises exposing at least one electromagnetic coil in such device to an external magnetic field and measuring current induced in such coil, wherein the coil surrounds or is surrounded by a flexible magnetic polymeric composite.

### BRIEF DESCRIPTION OF THE DRAWING

FIG. 1 is a plan view of a navigable guide catheter provided with a plurality of electromagnetic location sensors on a single core;

FIG. 2 is a sectional view of an electromagnetic location sensor taken along line 2-2′ in FIG. 1;

FIG. 3 is a plan view of the distal end segment of the FIG. 1 catheter in a bent position;

FIG. 4 is a plan view of a distal end segment of a navigable guide catheter provided with a plurality of electromagnetic location sensors on individual cores;

FIG. 5 and FIG. 6 are sectional views of two additional electromagnetic location sensors;

FIG. 7 is a perspective view of a location sensor bobbin; and

FIG. 8 through FIG. 9 are bar graphs showing mechanical and magnetic properties for an unfilled polymer and various flexible magnetic polymeric composites.

### DETAILED DESCRIPTION

The following detailed description describes certain embodiments and is not to be taken in a limiting sense. All weights, amounts and ratios herein are by weight, unless otherwise specifically noted. The terms shown below have the following meanings:

The term “elastomeric” when used in reference to a material means that the material, if stretched to at least 200% of its original length and released, will return with force to substantially its original length.

The term “flexible” means bendable. A flexible device may be resiliently bendable (viz., returning to or nearly to its original configuration when bent and then released) or deformably bendable (viz., remaining in or nearly in a bent configuration when bent and then released).

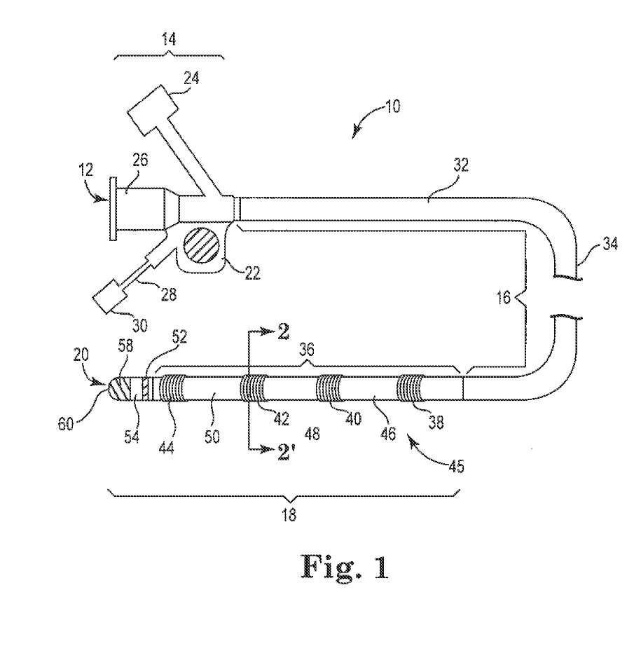

FIG. 1 is a plan view of a navigable, steerable open end guide catheter 10 including proximal end 12, proximal end segment 14, intermediate segment 16, distal end segment 18 and distal end 20. Proximal end segment 14 includes manipulative handle 22, shielded connector 24, access hub 26 and pull wire 28 with grip 30. Handle 22 is joined to elongated member 32 which surrounds pull wire 28 and other elements discussed in more detail below, and whose outer wall 34 may be made for example from a polyurethane, silicone or other biocompatible polymer suitable for use on the exterior of an insertable or implantable medical device. Distal end segment 18 includes a single flexible polymeric composite core 36 provided with a plurality of electromagnetic location sensing coils 38, 40, 42 and 44 wound around core 36 and separated from one another by unwrapped core portions 46, 48 and 50. More or fewer sensing coils than those shown in the embodiment depicted in FIG. 1 may be employed, and the sensing coils may have similar or different constructions. In the embodiment shown in FIG. 1, the sensing coils all have a similar construction. Core 36 may be made from a medically acceptable polymer within which magnetizable particles (not shown in FIG. 1) are dispersed. The type and loading level (viz., wt. %) of magnetizable particles desirably is sufficient to improve one or more performance-related sensor factors such the minimum required coil diameter, minimum required coil length, minimum required number of wire turns, the sensor or coil flexibility, or other factors influenced by the physical or electromagnetic characteristics of core 36 or sensing coils 38, 40, 42 and 44. Core 36 desirably has greater magnetic permeability than outer wall 34, thereby permitting a reduction in the required coil diameter or length or the required number of wire turns compared to sensing coils formed without such a core, e.g., sensing coils formed by wrapping wire around an outer wall 34 made from an unfilled polymer or from a polymer containing non-magnetically permeable material. Core 36 desirably is sufficiently flexible to facilitate insertion and navigation of catheter 10 through confined areas or tortuous paths within a patient undergoing surgery or treatment, and desirably has greater flexibility than a comparison device having sensing coils formed by wrapping wire around a solid or powdered magnetically permeable core. Flexing of core 36 may for example take place along any or all portions of core 36, e.g., along lengths of core 36 covered by wire turns, along lengths of core 36 not covered by wire turns, or along all portions of core 36. Core 36 desirably also is more flexible than intermediate segment 16, as this may facilitate bending or otherwise flexing distal end segment 18 rather than intermediate segment 16 as catheter 10 is advanced into a patient.

Distal end segment 18 may also include a generally ring-shaped anchoring member 52 encircling the outer circumference of sleeve 54 near the distal end 20 of catheter 10. Pull wire 28 may be fixedly attached to anchoring member 52, using for example welding or other appropriate bonding or joining methods. Anchoring member 52 may optionally serve as an electrode with pull wire 28 serving as a conductive element to carry electrical current between anchoring member 52 and a contact or other fitting in proximal connector 24. Distal end segment 18 also may include end cap member 58 equipped with central opening 60.

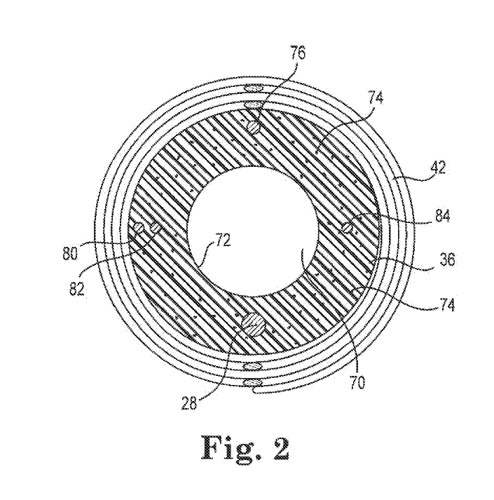

Opening 60 may communicate with one or more generally central lumens (such as the single central lumen 70 shown in FIG. 2) extending axially within elongated member 32 and thence with access hub 26 such that a medical device or therapy may be delivered through hub 26 and the central lumen(s) and may exit opening 60. End cap member 58 may be formed from a biocompatible polymeric material and may be over-molded onto distal end 20 of catheter 10. End cap member 58 may if desired be formed from a conductive biocompatible metal or alloy, for example stainless steel, platinum, iridium, titanium, or alloys thereof, and may serve as an electrode for sensing cardiac or other electrophysiological signals or for delivering current to a treatment site.

FIG. 2 shows a sectional view taken along line 2-2′ in FIG. 1. Central lumen 70 is defined by inner wall 72 of core 36. Magnetically permeable particles 74 are generally uniformly distributed throughout core 36. Pull wire 28 may as noted above be connected at its distal end to anchoring member 52. Electrode conductor 76 may for example be connected to conductive end cap member 58 or to another conductive surface (not shown in FIG. 1) at or near-the distal end of catheter 10. Conductors 80 and 82 may for example be connected to the respective distal and proximal ends of sensing coil 44. Conductor 84 may for example be connected to the distal end of sensing coil 42. Sensing coil 42 may as shown in FIG. 2 have several layers of wire surrounding core 36, or may have more, fewer or even a single layer of wire.

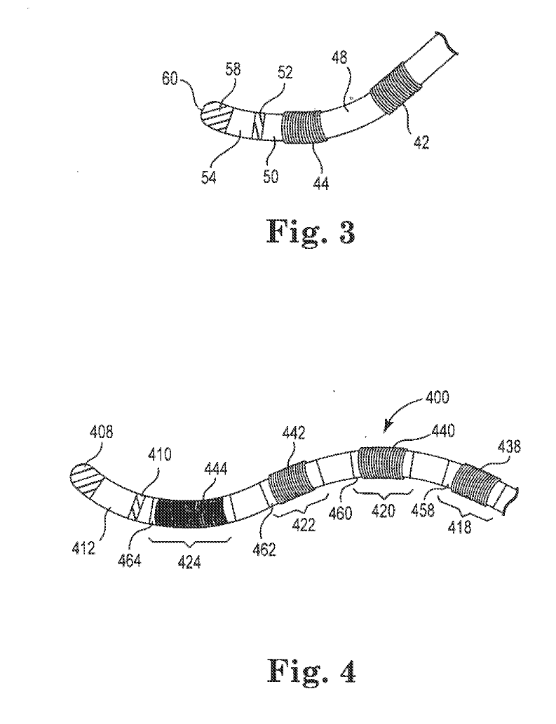

FIG. 3 is a plan view of a portion of the distal end of catheter 10 in a bent position. Bending may be restricted at coils 42 and 44, and less restricted at unwrapped core portions 48 and 50.

FIG. 4 is a plan view of a portion of the distal end of a closed end navigable guide catheter 400. Catheter 400 includes a tip 408, a generally ring-shaped anchoring member 410 encircling the outer circumference of sleeve 412, and sensors 418, 420, 422 and 424. Sensor 418 is formed by sensing coil 438 on core 458. Sensor 420 is formed by sensing coil 440 on core 460. Sensor 422 is formed by sensing coil 442 on core 462. Sensor 424 is formed by sensing coil 444 on core 464. More or fewer sensors than those shown in FIG. 4 may be employed, and the sensors may have similar or different constructions. In the embodiment shown in FIG. 4, sensors 418, 420 and 422 have similar constructions and sensor 424 (the most distally-located sensor) has a different construction. A device having a group of sensors including one different sensor such as sensor 424 need not deploy the different sensor in the most distally-located sensor position, and may instead deploy the different sensor in the most proximally-located sensor position or anywhere in between the most distal and most proximal sensor locations. Although each of sensors 418, 420, 422 and 424 is flexible, sensor 424 may have a more flexible construction than sensors 418, 420 and 422. Doing so may make it easier to bend sensor 424 and thereby aid in steering catheter 400 within a patient. Such more flexible construction may be accomplished in a variety of ways, including using fewer overlapping turns of wire (e.g., using a narrower or shorter core), more widely spaced turns of wire, thinner wire or more flexible wire in coil 444 compared to coils 438, 440 and 442; by using one or both of a lower loading of magnetically permeable particles or a more flexible polymer in core 464 compared to cores 458, 460 and 462; by using one or both of a larger inside diameter or smaller outside diameter for core 464 than for cores 458, 460 and 462; or by using a bellows-like construction, weakening lines, varying wall thickness or other flexibility-inducing measures to make core 464 more flexible than cores 458, 460 and 462.

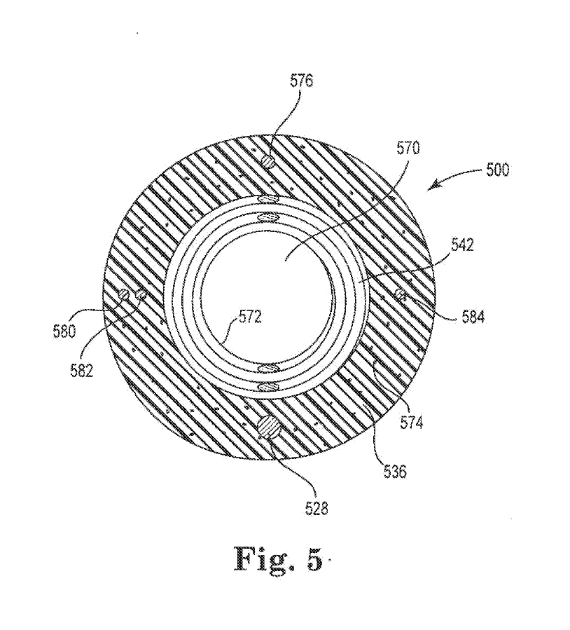

FIG. 5 shows a sectional view of a sensor 500 for use in the disclosed insertable or implantable medical devices. Sensor 500 includes coil 542 wound inside core 536. Central lumen 570 is defined by the inner wall 572 of coil 542. A protective polymeric coating (not shown in FIG. 5) may be applied to inner wall 572 to prevent damage to the wire insulation in coil 542. Magnetically permeable particles 574 are generally uniformly distributed throughout core 536. Pull wire 528 and conductors 576, 580, 582 and 584 may all pass through core 536.

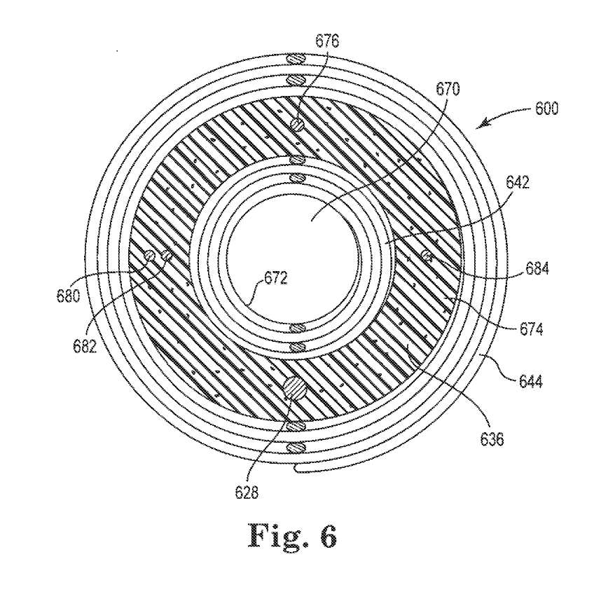

FIG. 6 shows a sectional view of a sensor 600 for use in the disclosed insertable or implantable medical devices. Sensor 600 includes coils 642 and 644 which are respectively wound inside and wrapped outside core 636. Central lumen 670 is defined by the inner wall 672 of coil 642. As in sensor 500, a protective polymeric coating (not shown in FIG. 6) may be applied to inner wall 672 to prevent damage to the wire insulation in coil 642. Magnetically permeable particles 674 are generally uniformly distributed throughout core 636. Pull wire 628 and conductors 676, 680, 682 and 684 may all pass through core 636.

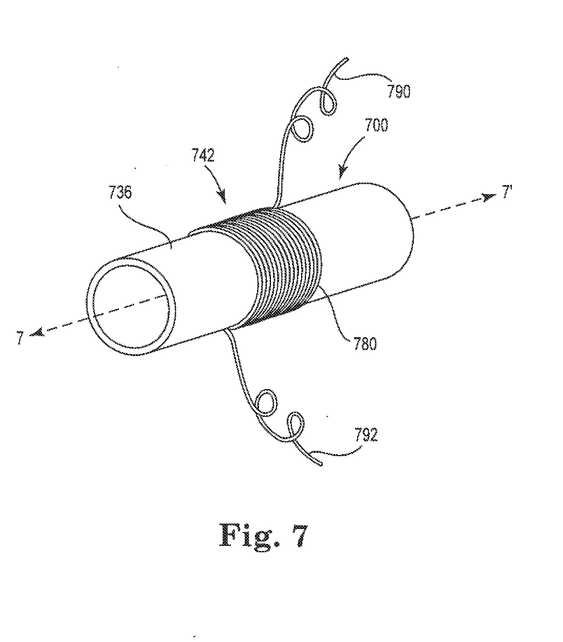

FIG. 7 is a perspective view of a location sensor bobbin 700 for use in manufacturing insertable or implantable medical devices. Bobbin 700 includes a discrete hollow cylindrical core 736 made from the disclosed flexible magnetic polymeric composite. Coil 742 is formed from fine-gauge insulated wire 780 wrapped around core 736. Coil ends 790 and 792 may be cut to an appropriate length and soldered or otherwise connected to suitable conductors, or may simply be left longer than shown in FIG. 7 and used as conductors in a later-formed insertable or implantable medical device (not shown in FIG. 7). Bobbin 700 is flexible and depending on the nature of the chosen magnetic polymeric composite may be resiliently or deformably bent with respect to its main axis of symmetry 7-7′.

A variety of polymers may be employed in the disclosed flexible magnetic polymeric composite, including polyamides (e.g., nylon rubbers), polyether amides (e.g., PEBAX™ block copolymer from Arkema), polyethylenes, fluoropolymers (e.g., polytetrafluoroethylene, polyvinylidene fluoride, and other polymers and copolymers of fluorinated monomers including DYNEON™ fluoropolymers from Dyneon LLC and TEFLON™ fluoropolymers from E. I DuPont de Nemours and Co.), polyimides, organosilicones and other silicone rubbers (e.g., SILASTIC™ elastomers from Dow Corning Corp.), polyurethanes (e.g., PELLETHANE™ thermoplastic polyurethane elastomers from Dow Chemical Co.), polyvinyl chloride, mixtures thereof, and other flexible polymeric materials which will be familiar to persons skilled in the field of insertable or implantable medical devices. Resiliently bendable cores may more readily be made by using elastomeric polymers, and deformably bendable cores may more readily be made by using elongatable but non-elastomeric polymers. The bending characteristics of a finished core may also be influenced by the chosen type and amount of magnetically permeable particulate materials.

A variety of magnetically permeable particulate materials may be employed in the disclosed flexible magnetic polymeric composite. The magnetically permeable material may for example be paramagnetic, ferromagnetic or ferrimagnetic, and may for example contain metals including iron, cobalt, nickel or gadolinium, used as is, alloyed with other metals, or used in oxide form and optionally combined with other oxides to form a variety of magnetically permeable ceramics. Desirably the magnetizable particles cause low or no hysteresis loss when the completed devices are used in a patient. Exemplary magnetically permeable materials include iron powder, carbonyl iron, magnetite, iron-silicon alloys, aluminum-nickel-cobalt (alnico) alloys, samarium-cobalt alloys, neodymium-iron-boron (NdFeB) alloys, ferrites, and other finely-divided magnetic particulates which will be familiar to persons skilled in the field of magnetically permeable materials. Exemplary commercially available magnetically permeable materials include HIPERCO™ 50 iron-cobalt-vanadium soft magnetic alloy, PERMALLOY™ nickel-iron magnetic alloys, PERNENDUR™ cobalt-iron and cobalt-iron-vanadium alloys and SUPERMALLOY™ nickel-iron-molybdenum alloys. The particles may for example have an average particle diameter of about 1 to about 100, about 2 to about 70 or about 10 to about 50 micrometers. Larger or smaller particles, including submicron particles or nanoparticles, may be used if desired for particular applications. The particles desirably have an average particle diameter less than about 20% of the core wall thickness. The particles may be surface-treated to improve their dispersibility in the magnetic polymeric composite. The magnetic polymeric composite desirably contains sufficient particulate material to increase induced current in the disclosed coil, compared to a device that does not contain such particulate material, when the coil is exposed to a fluctuating applied external magnetic field. The magnetic polymeric composite may for example contain about 2 to about 60, about 5 to about 50 or about 10 to about 50 volume % particles. The addition of magnetically permeable particles may also affect, sometimes adversely, other composite physical properties (for example, ultimate tensile strength, strain at yield or elongation at yield) and accordingly it may be desirable to strike a balance between an increase in magnetic permeability and a potential decrease in other physical properties. Relatively small additions of magnetically permeable particles can provide very desirable overall performance. For example, an addition of about 20 volume % of 10 micrometer average diameter SUPERMALLOY nickel-iron-molybdenum alloy particles to PEBAX block copolymer can provide an appreciable increase in magnetic permeability while maintaining other desirable physical properties such as ultimate tensile strength and strain at yield.

The core may comprise, consist essentially of or consist of the disclosed polymer and magnetically permeable particles. The core may if desired contain a variety of adjuvants, including fillers, extenders, radioopacifying agents (e.g., radioopacifying fillers), surface-active agents, polymer processing aids, pigments, and other ingredients which may improve the performance or processability of the magnetic polymeric composite.

The magnetic polymeric composite may be processed to form cores in a variety of ways including extrusion, pressure molding, dip coating and other techniques including those discussed in U.S. Pat. No. 5,817,017 to Young et al., for example by extrusion at or above the polymer melt flow temperature. The resulting cores may have a variety of shapes. For example, the core may have a cylindrical shape with coils wrapped around the outside of all or part of the cylinder sidewall, or with coils wound inside all or part of the cylinder sidewall. The core may also have a toroidal shape with coils wrapped entirely or partially around the toroid surface.

The wire in the disclosed coils may be made from a variety of materials including copper, gold and other medically acceptable metals or alloys which will be familiar to persons skilled in the field of insertable or implantable medical devices. The wire may be any type and diameter suitable for formation of sufficiently compact and durable coils, e.g., varnish-or otherwise-insulated wire in American Wire Gauge (AWG) sizes 58 (0.01 mm or 0.0004 in) to 38 (0.1 mm or 0.004 in). Larger or smaller diameter wire may be used if desired for particular applications. The coil may have a variety of lengths, for example a length of about 1.27 mm (0.05 in) to about 6.35 mm (0.25 in). The coil length desirably is less than about 2.5 mm (0.1 in). The coil may cover all or only a portion of the core. In one exemplary embodiment the core is about two to about three times (e.g., about 2½ times) as long as the coil along the central core axis. The number of coil turns may vary, and may for example be about 33 to about 167 turns per layer (e.g., about 66 turns per layer) for a four layer coil having a 2.5 mm length. The coil may be wound in a single layer or in a plurality of layers, with a low number of layers being desirable where reduced outer diameter or increased inner diameter are desired, for example to permit use of a smaller device in small blood vessels, to reduce recovery time, or to accommodate space for additional features in an existing device. In some embodiments, coils having fewer than 100 turns may be employed. Other numbers of turns and wire diameters may be employed depending on the desired sensor application. Exemplary coil configurations include those shown in U.S. Pat. No. 5,727,552 to Saad, U.S. Pat. No. 6,385,471 B2 to Hall et al. and U.S. Pat. No. 7,130,700 B2 to Gardeski et al., in U.S. Patent Application Publication No. US 2004/0097806 A1 to Hunter et al. and in published International Patent Application No. WO 99/40957 A1. A suitably thin and optionally flexible coating may be applied to the finished coil to help hold the wire in place when the core is bent or to help prevent damage to insulation on the coil wire.

The outermost portion of the core and coil may have a variety of diameters. Exemplary maximum diameters for the core, coil or for the distal end of the disclosed devices are for example at least about 1 French (0.33 mm or 0.013 in) and less than or equal to about 10 French (3.3 mm or 0.131 in), 9 French (3 mm or 0.118 in), 8 French (2.7 mm or 0.105 in), 7 French (2.3 mm or 0.092 in), 6 French (2 mm or 0.079 in), 5 French (1.67 mm or 0.066 in), 4 French (1.35 mm or 0.053 in) or 3 French (1 mm or 0.039 in).

The core and coil may be designed with the aid of equation I shown below:

$$L = π \times μ \times \frac{( {n}^{2} \times {r}_{ave}^{2} )}{l} \qquad I$$

where: L is the induced current,
μ is the magnetic permeability of the core material,
n is the number of wire turns,
rₐᵥₑ is the effective radius of the coil, and

l is the length of the coil.

In general, it is desirable to produce the largest signal possible in the coil so that the coil position in space can be determined with less error or less signal-to-noise ratio. However, as rₐᵥₑ decreases, the current induced in the coil decreases exponentially. Increasing the number of wire turns can provide an offsetting exponential increase in induced current. However, this may increase the coil length l and thereby undesirably increase coil rigidity. Through appropriate selection of the magnetic polymeric composite and the type and loading level of magnetically permeable particles, the core permeability μ may be increased sufficiently to permit downsizing the coil radius or changing the core or coil construction in other ways without sacrificing flexibility, minimum turn radius or other relevant steering or navigation properties for an insertable or implantable medical device.

The disclosed coils and cores may be used in a variety of insertable or implantable medical devices, including catheters (e.g., open-ended or close-ended ablation catheters, balloon catheters, stent delivery catheters, electrophysiological diagnostic catheters, pressure monitoring catheters, biologic delivery systems, and intravascular imaging devices such as intravascular ultrasound or IVUS and intracardiac echocardiography or ICE), leads (e.g., cardiac pacing, cardiac defibrillation cardiac or neurological leads), endoscopes, biopsy tools and other elongated medical devices. The distal ends of such devices may have a variety of shapes including ball ends, tapered ends and blunt ends. The devices may have no lumen, a single lumen or multiple lumens. The devices may include splined bodies (e.g., as shown in the above-mentioned U.S. Pat. No. 7,130,700 B2), and if desired all or a portion of such splined bodies may be made from the disclosed magnetic polymeric composite. The devices may include other components employed in insertable or implantable medical devices, for example pull wires, guide wires or stylets, electrodes, conductors, fluid delivery or other needles, additional sensors, deflection members, selectively activated shape memory devices and other components such as those discussed in the above-mentioned U.S. Pat. No. 7,130,700 B2. The disclosed devices may be steered or located in a patient using a variety of equipment and techniques including those discussed in U.S. Pat. No. 5,983,126 to Wittkarnpf and published International Patent Application No. WO 01/24685 A2.

The invention is further illustrated in the following non-limiting examples in which all parts and percentages are by weight unless otherwise indicated.

### Example 1

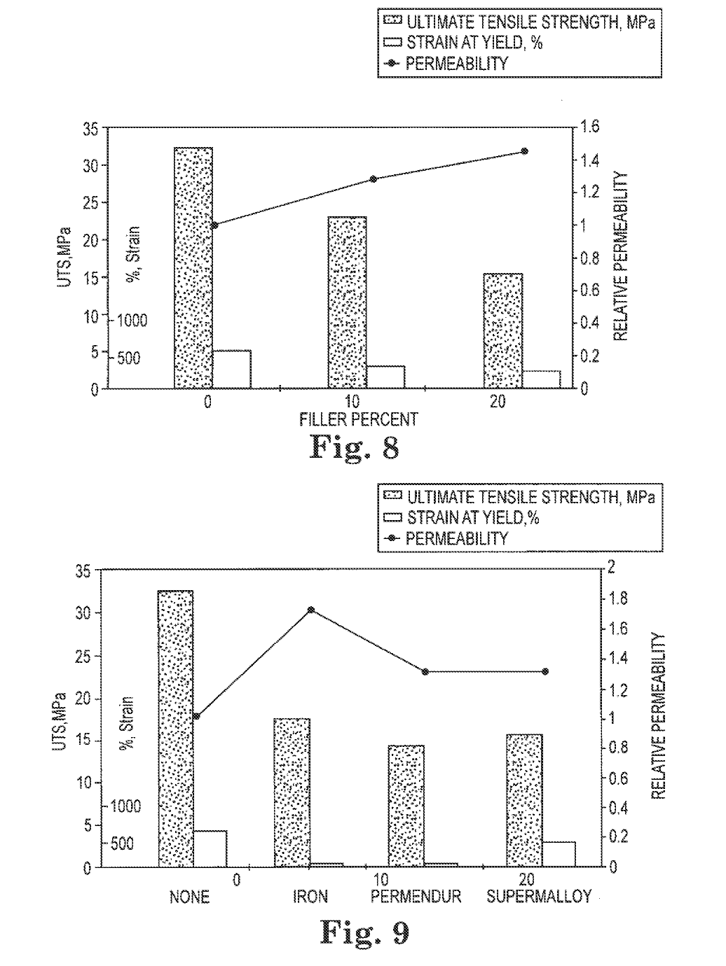

A sample of PEBAX 55D block copolymer from Arkema was compounded in a batch mixer with 22 micrometer average diameter SUPERMALLOY particles (from Ultrafine Powder Technology, Inc., Woonsocket, R.I.) at 0 and 20% loading levels. The ultimate tensile strength, strain at yield and magnetic permeability for the resulting composites are shown in FIG. 8 together with the results obtained for the unfilled copolymer. The magnetic permeability value increased from 1 to more than 1.4 as the loading level increased from 0 to 20%. The increased permeability observed at a 20% loading level should enable rₐᵥₑ , the effective coil radius, to be reduced by about 18% or more while still maintaining a comparable level for L, the induced current.

### Example 2

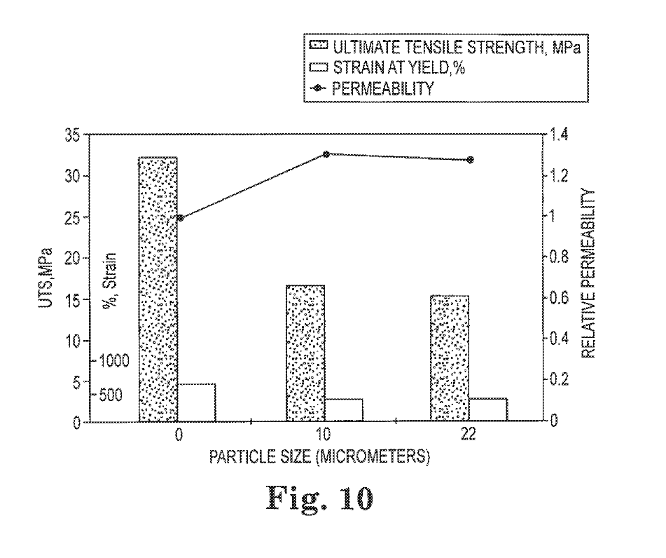

Using the method of Example 1, PEBAX block copolymer was compounded with 22 micrometer average diameter iron particles (from Atlantic Equipment Engineers, Bergenfield, N.J.), PERMENDUR cobalt-iron-vanadium alloy particles (from Ultrafine Powder Technology, Inc.) or with SUPERMALLOY nickel-iron-molybdenum alloy particles (from Ultrafine Powder Technology, Inc.), at a 20 volume % loading level. The ultimate tensile strength, strain at yield and magnetic permeability for each of the resulting composites are shown in FIG. 9 together with the results obtained for the unfilled copolymer. The magnetic permeability value when using iron particles was more than 1.7, and the magnetic permeability value when using the alloys was about 1.3. These increases in magnetic permeability should enable rₐᵥₑ to be reduced by 30% when using iron or by about 14% when using the alloys while still maintaining a comparable level for L.

### Example 3

Using the method of Example 1, PEBAX block copolymer was compounded with 10 and 22 micrometer average diameter SUPERMALLOY particles (from Ultrafine Powder Technology, Inc.), at a 20 volume % loading level. The ultimate tensile strength, strain at yield and magnetic permeability for each of the resulting composites are shown in FIG. 10 together with the results obtained for the unfilled copolymer.

Although specific embodiments have been illustrated and described herein for purposes of description of the preferred embodiments, it will be appreciated by those of ordinary skill in the art that a wide variety of alternate or equivalent implementations calculated to achieve the same purposes may be substituted for the specific embodiments shown and described without departing from the scope of the present invention. This application is intended to cover any adaptations or variations of the preferred embodiments discussed herein. Therefore, it is manifestly intended that this invention be limited only by the claims and the equivalents thereof.

We claim:

1. An insertable or implantable medical device comprising an elongated member having a proximal end, a distal end, at least one conductive coil near the distal end, and electrical conductors which carry current from the coil towards the proximal end, wherein the coil surrounds or is surrounded by a flexible magnetic polymeric composite.

2. A device according to claim 1 wherein the magnetic composite is elastomeric.

3. A device according to claim 1 wherein the magnetic composite comprises a polyamide, polyether amide, polyethylene, fluoropolymer, polyimide, organosilicone, polyurethane, polyvinyl chloride or mixture thereof.

4. A device according to claim 1 wherein the magnetic composite comprises magnetically permeable particulate material.

5. A device according to claim 4 wherein the particulate material comprises iron, cobalt, nickel or gadolinium.

6. A device according to claim 4 wherein the particulate material comprises a ceramic.

7. A device according to claim 4 wherein the particulate material comprises iron powder, carbonyl iron, magnetite, iron-silicon alloy, aluminum-nickel-cobalt alloy, samarium-cobalt alloy, neodymium-iron-boron alloy, ferrite or mixture thereof.

8. A device according to claim 4 wherein the magnetic composite comprises sufficient particulate material to increase induced current in the coil, compared to a device that does not contain such particulate material, when the coil is exposed to a fluctuating applied external magnetic field.

9. A device according to claim 4 wherein the magnetic composite comprises about 2 to about 60 volume % particulate material.

10. A device according to claim 4 wherein the particulate material has an average particle diameter of about 1 to about 100 micrometers.

11. A device according to claim 1 wherein the coil surrounds the magnetic composite and the magnetic composite is hollow.

12. A device according to claim 1 wherein the coil comprises an electromagnetic location sensor.

13. A device according to claim 1 wherein the device comprises a plurality of coils.

14. A device according to claim 13 wherein the coils have similar construction.

15. A device according to claim 13 wherein at least one coil has different construction from the remaining coils.

16. A device according to claim 13 wherein at least one coil is more flexible than the remaining coils.

17. A device according to claim 16 wherein a more flexible coil is nearer the distal end than a less flexible remaining coil.

18. A device according to claim 1 wherein the coil is wound in a single layer.

19. A device according to claim 1 wherein the coil is wound in a plurality of layers.

20. A device according to claim 1 wherein the coil has a length of about 1.27 mm to about 6.35 mm.

21. A device according to claim 1 wherein the coil comprises wire having a diameter of about 0.01 mm to about 0.1 mm.

22. A device according to claim 1 wherein the coil has a central axis and the magnetic composite and coil are resiliently bendable along such axis.

23. A device according to claim 1 wherein the coil has a central axis and the magnetic composite and coil are deformably bendable along such axis.

24. A device according to claim 1 whose distal end has a maximum diameter less than or equal to 10 French.

25. A device according to claim 1 having at least one lumen.

26. A device according to claim 1 having a plurality of lumens.

27. A device according to claim 1 comprising a catheter.

28. A device according to claim 1 comprising a lead.

29. A device according to claim 1 comprising an endoscope.

30. A location sensor bobbin comprising a conductive coil surrounding or surrounded by a flexible magnetic polymeric composite, the bobbin being hollow and being sized and shaped to fit on or into an elongated insertable or implantable medical device.

31. A method for making an insertable or implantable medical device, which method comprises forming an elongated member having a proximal end and a distal end, forming at least one conductive coil surrounding or surrounded by a flexible magnetic polymeric composite near the distal end, and connecting electrical conductors to the coil to carry current from the coil towards the proximal end.

32. A method for locating an elongated insertable or implantable medical device in a patient, which method comprises exposing at least one electromagnetic coil in such device to an external magnetic field and measuring current induced in such coil, wherein the coil surrounds or is surrounded by a flexible magnetic polymeric composite.
